(19) United States
(12) Patent Application Publication
(10) Pub. No.: US 2006/0287590 A1
(43) Pub. Date: Dec. 21, 2006

(54) Noninvasive vital sign measurement device

(76) Inventor: Edwin L. McEowen, Columbia City, IN (US)

Correspondence Address: BARNES & THORNBURG LLP, 600 ONE SUMMIT SQUARE, FORT WAYNE, IN, 46802, US

(21) Appl. No.: 10/944,161

(22) Filed: Sep. 17, 2004

Related U.S. Application Data
(60) Provisional application No. 60/504,295, filed on Sep. 18, 2003.

(51) Int. Cl. A61B 5/00; A61B 6/00; A61B 5/02

(52) U.S. Cl. 600/331; 600/368; 600/474; 600/485

(57) ABSTRACT

A blood monitoring device comprising a body having a proximal and distal transducers spaced apart and connected to both device drivers and recognition and analysis devices. A power source is connected to both device drivers and the recognition and analysis devices. The device driver is also connected to the recognition and analysis devices.

This application claims priority based on U.S. Provisional Patent Application Ser. No. 60/504,295, entitled “Noninvasive Vital Sign Measurement Device,” and filed Sep. 18, 2003.

### BRIEF DESCRIPTION OF THE DRAWINGS

FIG. 1 is a diagrammatic view of a body part having the new and improved noninvasive vital sign measurement device of the invention attached thereto utilizing a spaced apart sender and receiver;

FIG. 2 is a view like FIG. 1 of another version of the new and improved noninvasive vital sign measurement device of the invention showing a single sender and receiver;

FIG. 3 is a view of another version of the new and improved vital sign measurement device using three or more transducers;

FIG. 4 us a view of still another version of the new and improved vital sign measurement device similar to that shown in FIG. 1; and

FIG. 5 is a view of still another version of the new and improved vital sign measurement device in which the transducers are mounted on opposite sides of a blood vessel or vessels.

### DESCRIPTION OF A SPECIFIC EMBODIMENT

The new and improved noninvasive vital sign measurement device 10 of the invention is a medical device for supplying vital sign measurements for any purpose and in any setting where such information is useful to medical clinicians conducting physical examinations or monitoring patients (inpatient, outpatient, or ambulatory), whether in well-equipped hospitals, clinics, or on a battlefield. The invention would allow the monitoring of vital signs in a noninvasive manner. In the vascular application of the device, vital signs that can be measured would include arterial and venous blood pressure and pulse, blood flow velocity, and blood density. Peripheral vascular resistance could be calculated and displayed using data from the device. More conventional equipment could be mated with the device in order to continuously monitor such things as temperature and oxygen saturation. Other potentially measurable pressure parameters could include the extravascular space, intracranial space, intrathoracic (vascular, airway, and pleural) space, or any confined body cavity, depending upon the particular configuration of the device and where it is mounted upon or applied to the body. Examples of confined body cavities would include possibly the urinary bladder, gallbladder, intra-abdominal, ocular, and more probably extremity fascial compartments. Additional measurements that may be obtainable by the device could be other vascular parameters including possibly intracardiac chamber pressures and more possibly central venous pressures.

When arterial blood pressure is measured and monitored, both systolic and diastolic blood pressure should be monitored beat-by-beat. This information would be useful in evaluating routine vital signs, hypertension, hypotension, and shock from any cause. The instantaneous monitoring by the application of the invention would provide a means by which the effectiveness of pharmaceutical intervention and surgical intervention could be immediately assessed. Venous and extravascular space monitoring can be used to determine tissue perfusion and lymphatic obstruction, as well as the general state of hydration of the patient. Vascular monitoring will provide information regarding patient shock from any cause, e.g., sepsis, blood loss, and autonomic malfunction. Data from the combined monitoring of arterial pressure and blood flow could be used to calculate vascular resistance. For the clinician, knowing the level of vascular resistance and continuously monitoring blood pressure are key factors in determining not only the cause of shock but also the best course of treatment in each circumstance.

Intrathoracic measurements could include intrapulmonary and intracardiac, as well as pleural and pericardial space pressures. Measurement of large, medium, and small airway and alveolar space pressures would give physicians both diagnostic and treatment monitoring tools for acute and chronic lung disease. The device could be used to confirm endotrachial tube placement. Intrapleural pressure measurements would provide data for rapid diagnosis or confirmation of hemothorax and pneumothorax, and could be used in both hospital and prehospital settings to help determine the urgency with which these conditions should be treated.

Intracardiac pressure measurements would allow diagnosis of valvular failure, cardiomyopathy, congenital defects, myocardial ischemia/infarct, and congestive hear failure. Chamber pressure measurements together with echocardiogram data and pulmonary vascular readings would yield vital information regarding the etiology of any of the above maladies previously available only with cardiac catheterization.

More convenient and accurate ocular pressure measurement would allow physicians improved means of diagnoses and treatment monitoring of ocular diseases such as glaucoma.

Intracranial pressure measurements would most likely be extremely difficult because of signal attenuation through bone; however, if possible, it would give physicians a rapid estimate of tissue pressure, ventricular pressure, and vascular space pressure when dealing with patients suffering from head injury or stroke, and post-operative neurosurgical patients. It would also be useful in the diagnosis of such maladies as pseudotumor cerebri and hydrocephaly.

Currently, most all of the measurements above can be obtained accurately only by the use of expensive and/or invasive procedures. Sphygmomanometer blood pressure cuff readings are accurate in normal and high ranges, but cumbersome and slow, as well as painful for many patients. For automatic blood pressure cuff devices, the International Electrotechnical Commission has set international standards regarding strict limits on the pressure to which the cuff can be inflated. And, in order to avoid tissue damage and considerable discomfort to the patient, they have also set limits on the period of rapid inflation/deflation cycles. Blood pressure cuff readings are in fact contraindicated for post-mastectomy patients in the arm on the affected side. However, when vital signs are unstable or potent drugs are needed in order to maintain blood pressure, time-consuming invasive procedures are required for continuous monitoring. In the emergent setting, clinical decisions must often be made long before there is any x-ray or echocardiography evidence available and long before invasive vascular monitoring catheters can be inserted and calibrated.

Vital sign data which can be obtained by the device are useful in intensive care units, operating rooms, all prehospital settings, emergency departments, dialysis centers, medical practice offices, medical research, pulmonary and veterinary clinics, in military installations or on a battlefield, and in aerospace installations for monitoring pilots and astronauts at work. On an ambulatory basis, such data would also be very useful in everyday life and in the sports world. We currently have no convenient way to monitor the businessman, the homemaker, or the athlete in action.

The function of the device depends upon subtle, but measurable, changes in acoustic velocity that occur as a result of changes in density of the medium through which the sound wave is propagating. The noninvasive device would measure acoustic transit times, and thereby measure density within fluid or gas-filled body organs/structures/vessels. By monitoring transit times and minute shifts in transit time in rapid sequence (10 to 100 times per second) during all phases of systole and diastole, such measurements, if made with precision, would result in accurate, reliable, and continuous vital sign data.

In addition to arterial and venous pressure readings, this principle would in like manner apply to the measurement of pressure in gas-filled structures such as pulmonary airways and possibly the bowel lumen. Similarly, ocular, intrauterine, and possibly extremity compartment pressures would be amenable to measurement. Intracranial pressures may be measurable with this technique as well. The acoustic frequency specifications and configuration of the device would be altered according to the purpose at hand; e.g., airway pressure measurement would require much lower frequencies for better intrathoracic sound penetration and since acoustic velocity is much slower in gas than in fluid.

The measurements are based upon the characteristics of acoustic waves as they propagate through biologic tissue or fluids or gases. Since acoustic velocity increases with the density of the medium through which it is propagating, then there must be a measurable change in acoustic velocity through a fluid or gas-filled vessel, cavity, or compartment as the density within changes. Minute blood density fluctuations will occur as the blood pressure cycles between systole and diastole. Therefore, there must be a measurable change in the acoustic wave propagation velocity through the blood as the pressure changes. The common equation, V=D/T, indicates that changes in velocity (V) are inversely proportional to changes in transit time (T) over a fixed distance (D). If the measurements were done with precision, then the device output would consist of highly accurate, beat-by-beat digital pressure readings in the case of vascular application of the device.

The UNESCO equation describes the relationship between acoustic wave velocity and pressure in water. The equation also takes into account other factors that contribute to the density of the fluid, such as the salinity and the temperature. Although fluids (blood included) are considered incompressible, the equation shows that there should be minute but measurable changes in velocity associated with changes in pressure, even within the human blood pressure range of 0 to 300 mmHg.

To form theoretical support for this method, the space between two hypothetical transducers was assumed to be 10 cm. The UNESCO equation was then used to calculate acoustic velocity at pressure increments of 10 mmHg assuming fluid temperature is 37 degrees Celsius, salinity is 9 psu (practical salinity units) or ppt (parts per thousand), and variable pressure is expressed in kPa. Calculations using the formula V=D/T indicate that in order for the device to have precision to within 1 mmHg, it must be capable of detecting shifts in transit time of roughly 10 picoseconds. Trending of the pressure could be achieved by the detection of shifts of approximately 100 picoseconds.

Referring now to FIG. 1, the monitoring device 10 is shown attached to a body part 12 from which the blood pressure and other vital signs are monitored. Body part 12 can be any body part including the head, the neck, the chest, the abdomen, the arms, and the legs, to measure pressure in any blood vessel in good proximity to the skin's surface. Two transducers 14, 16 are spaced apart, longitudinally in line with a vessel, a specific and fixed distance, e.g., 10 cm, and applied to the skin using an acoustic conductive medium. By measuring the transit time of the acoustic signal between the two transducers 14, 16, the velocity of the sound wave through the tissue can be calculated using the equation V=D/T, where V equals the velocity; D equals the space between the transducers 14, 16; and T equals the time the signal takes to propagate (transit time) between the two transducers 14, 16. One transducer 14 (the sender) generates the input signal and the other transducer 16 (the receiver) generates the output signal.

Utilizing this pitch-catch method, with the two transducers 14, 16 both serving the dual function of sender and receiver, measurements of both upstream and downstream transit times would be achieved. Blood flow velocity would be calculated in a conventional manner using the difference between downstream and upstream transit times. Since transit time oscillations resulting from blood flow are magnitudes greater than transit time oscillations associated with cyclical pressure changes, these flow oscillations must be effectively cancelled out of the calculation by the summation of downstream and upstream transit times. The data resulting from this summation would reflect the effect of pressure fluctuations on transit times. The summation would magnify the observed systolic/diastolic shift in transit times by a factor of two while canceling the effect of blood flow. Also, this technique would reduce artifact resulting from body movement and intravascular turbulence. Mathematically this can be expressed as follows:
Tₜₒₜₐₗ=(T_{downstream}+Tᵤₚₛₜᵣₑₐₘ)/2

Factors determining total transit time are (1) acoustic velocity due to blood density, (2) acoustic velocity as it is influenced by blood flow and artifact produced by body movement and vascular turbulence, and (3) the velocity of the acoustic wave as it passes through the surface conductive medium and the skin and subcutaneous tissues.
Vₜₒₜₐₗ=V₁+V₂+V₃

V₃ (tissue and conductive medium contribution to velocity) will remain constant. V₂ (blood flow and artifact contribution to velocity) can be readily measured and canceled out of the equation by summing the velocity in both directions, thereby eliminating its contribution to the equation. Therefore, V₁ (density contribution to velocity) remains as the only variable factor when transit times are measured, and, within blood vessels, pressure will be the only density determining factor that fluctuates on a moment by moment basis.
Vₜₒₜₐₗ=V_{density}+V_{constant}

Therefore:
ΔVₜₒₜₐₗ=ΔV_{density}

Since density—as it is determined by the momentary values of hematocrit, salinity, and temperature—remains fixed, then it follows that any momentary velocity fluctuations are a result of fluctuations in pressure alone. Thus:
ΔV_{density}=ΔVₚᵣₑₛₛᵤᵣₑ
and therefore:
ΔVₜₒₜₐₗ=ΔVₚᵣₑₛₛᵤᵣₑ

Therefore, any momentary fluctuation in transit times will also be a result of fluctuations in pressure. These fluctuations in transit times can thus be expressed mathematically as:
ΔTₜₒₜₐₗ=ΔTₚᵣₑₛₛᵤᵣₑ
and since:
ΔTₜₒₜₐₗ=(ΔT_{downstream}+ΔTᵤₚₛₜᵣₑₐₘ)/2
then therefore:
ΔTₚᵣₑₛₛᵤᵣₑ=(ΔT_{downstream}+ΔTᵤₚₛₜᵣₑₐₘ)/2

Signal processing and adequate sound conduction through the skin and subcutaneous tissues to and from the structure of interest are critical steps involved in ensuring the accuracy and reliability of the device. Factors such as incorrect device placement, obesity, and edema will interfere with acoustic conduction and possibly render the device ineffective. In an aerospace application or during sports participation, high G forces may effectively dislodge the device from its proper position. The use of bi-directional “pitch-catch” transducers will reduce the error resulting from imprecise device placement upon the body. This method will also likely reduce artifact from body movement.

The output signal would appear as an amplitude spike (buried within noise) that moves to and fro along the instrument's time scale indicating at its limits a systolic and diastolic transit time for each cycle. Shorter transit times are associated with the systolic pressure and longer transit times with the diastolic pressure. The point along the larger transit time scale where these minute pressure-related transit time shifts will be observed will drift as blood density drifts due to changing physiologic values such as hematocrit, salinity, and temperature. This drifting must be accounted for by the continuous and precise monitoring of blood density as it is affected by these varying physiologic values. This can be done using the precision acoustic transit time measurements described earlier, and it is essential for continuous calibration of the device as base-line drifting of blood density occurs.

Ideally, calibration measurements will be made by observing the density of venous blood while it is under the influence of zero increased vascular pressure, i.e., at atmospheric or ambient pressure. As the device operates in its vascular mode, continuous calibration and ultra-precision is the core of its design and function. To summarize, the core of the device design and function depends upon ultra-precision and continuous calibration for changes in temperature and also for changes in transducer separation distance (if not fixed by use of a rigid housing). (See page 34.)

According to the UNESCO equation, there is a 98.4 picosecond change in the transit time across a 10 centimeter distance for every 10 mmHg change in pressure. However, the variability of the above mentioned physiologic values may result in as much as several hundred nanoseconds of drift in transit times. According to the UNESCO equation, when salinity is fixed at 9 psu, a change in body temperature of 1 degree Celsius would alter transit time by about 75 nanoseconds (a scale that is magnitudes greater than transit time shifts resulting from incremental changes in pressure). Similarly, with temperature fixed at 37 degrees Celsius, alterations in salinity of only 0.1 psu would result in a change in transit time of about 4.25 nanoseconds. These values were calculated using velocity data obtained from the UNESCO equation at the National Physical Laboratories (NPL) interactive website and using the previously noted common equation, V=D/T. Such large scale changes in blood density would of course occur over a period of hours and not milliseconds and therefore should not affect momentary pressure readings. However, if blood density alterations are not monitored precisely and continuously, then the minute fluctuations in transit time related to pressure oscillations would have no baseline or frame of reference and would be useful only for pulse detection. Even trend monitoring would be difficult as such without a solid frame of reference.

Ideally, in order for the peak and trough (systolic and diastolic) density values to be meaningful, the measurement of baseline density must be performed within the observed fluid or gas when it is under zero increased pressure. In vivo, however, blood or other physiologic fluids or gases are rarely without the influence of at least minimal pressure. This fact increases the challenge of device calibration. However, it can be predicted intuitively that there may be a measurable “zero” or baseline density that could be monitored by the device by selectively “capturing” venous system readings during the lowest point in the cycle (most likely during inspiration at end-diastole). It can also be predicted that there may be a mathematical relationship between peak and trough arterial and venous density and flow values and the baseline density value. This prediction allows for the potential determination of the baseline calibration density by means of extrapolation. Another means of device calibration, much less desirable because of its semi-invasive nature, would be the measurement of the density of an in vitro blood sample at atmospheric pressure.

The best method for ultrasound (US) or electromagnetic (EM) pulse delivery and detection must be determined. Potential devices would include conventional high frequency ceramic piezoelectric US transducers, RF (radio frequency) US transducers, polymer piezoelectric US transducers, IR (infrared) receivers, and Fiber Bragg Grating (FBG) Laser receivers, not excluding other existing and/or future transducers or sensors which are found to be applicable. All of these devices are referred to herein as “transducers” and/or “sensors.” The frequency and amplitude chosen for the input signal, as well as the mechanism of its delivery, will depend upon requirements for patient safety and requirements for proper tissue penetration and conduction of the acoustic wave.

The device must be capable of detecting transit time shifts as low as 9.8 picoseconds in order for it to have resolution of 1 mmHg pressure, which would be ideal for medical purposes. Medical ultrasound typically operates in the frequency range of 1 to 10 MHz. This device will likely require a higher frequency acoustic input signal for accuracy. However, lower frequencies better penetrate tissues with less attenuation.

Input signal attenuation and penetration varies between tissue types and according to the frequency. For example, according to Dowsett, Kenny and Johnston: The Physics of Diagnostic Imaging, chapters 17, 18; attenuation coefficient/frequency (dBcm-1 Hz-1) are listed for the following tissue types: muscle: 1.8-3.3; fat: 0.6; brain: 0.9; blood: 0.2; bone: 20. These variations in signal attenuation can be exploited in order to enhance the quality of the output signal, since blood is a better conductor of acoustic energy and less prone to signal attenuation when compared to biologic tissues. However, signal attenuation is much higher at high frequencies. Nevertheless, there exists enough of a difference between its value in blood and tissues that the principle remains the same. Ideally, the chosen frequency would attenuate within the skin and subcutaneous tissue before directly reaching the receiver yet conduct effectively along the vessel to the receiver. This would greatly enhance the signal-to-noise ratio.

Signal input from the sender must consist of brief pulses or “clicks” generated at specific intervals (e.g., 10 to 100 times per second) in order to detect all phases of the pressure cycle. The brevity of the impulse will be important for precision and will guide the choice of acoustic energy to be considered for use in the device. There will likely be a need to focus the ultrasound beam in such a way as to effectively maximize the intravascular acoustic travel distance (the distance that the sound wave actually travels within the blood vessel on its path to the receiving transducer). Such focusing will probably take the form of a simple transducer array, possibly requiring the use of more than one frequency.

Also for the sake of precision, the operational goal of device 10 is for the sending transducer to create a focused shock wave “click,” and to clock its transit time within the blood to the receiving transducer. Since sound waves travel in all directions from their point of origin, it would be difficult to know the exact length of the intravascular sound wave path. However, fixing the transducer separation by using rigid housing and using a technique such as Time-Reversal Mirrors should define the wave path well enough to accomplish the desired level of precision. Maximizing and closely defining the length of the sound wave path is a crucial step for the accurate determination of intravascular sound speed. Detection and timing of the first arrival wave would indicate the transit time for the most direct path between transducers. Since sound speed is higher in blood than in the surrounding tissues, then this first arrival wave would be considered to have passed through blood.

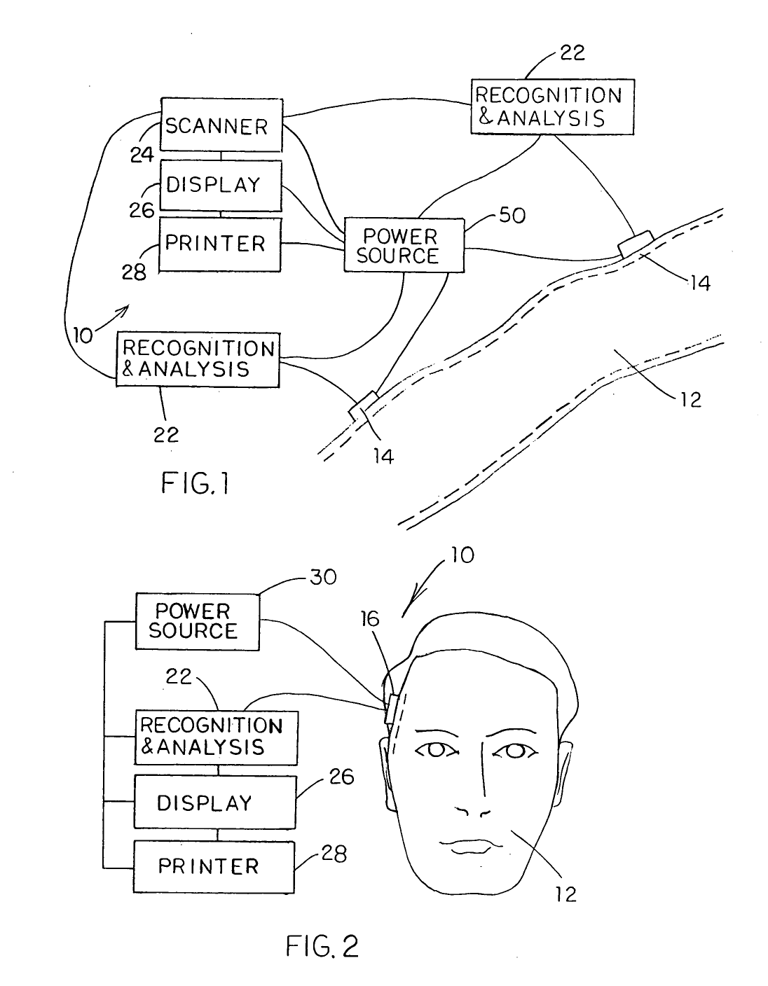

Referring to FIG. 2, there is shown the device 10 of the invention in a single transducer variant comprising a transducer 18 applied to the skin of a body part 12 using an acoustic conductive medium such as above described. The single transducer functions as the sender, generating the acoustic input signal and as the receiver, generating the output signal. The single transducer 18 could yield the same data as the two-transducer method, above described. This may be accomplished by measurement of the transit time (the echo) of the acoustic wave to and from the far side of the specific vessel as the wave reflects off the vessel wall interface. Using this method, the angle of the transducer axis to the vessel is critical. The transducer must remain as close to perpendicular as possible to the plane of the vessel in order to eliminate errors caused by blood flow. As this method may not be as precise as the two-transducer method, it may be more useful for trending.

Phase-shift detection could be used as another signal processing technique in both the single and two-transducer methods to detect transit time shifts in vessels, chambers, body cavities and compartments, or airways. Since the velocity of acoustic transmission changes with varying pressure, the phase of the reflected or transmitted wave would shift proportionately with changes in transit time and therefore would also shift with changes in pressure. When using this phase-shift detection technique during vascular system or static fluid compartment measurement, very high frequencies (most likely within the range of 50 MHz to 7.5 GHz, but not excluding higher or lower frequencies) would be required in order to ensure precision. When analyzing the pulmonary or pleural spaces, lower frequencies (probably ranging from 100 KHz to 1 MHz, but not excluding higher or lower frequencies) would be required.

Such a phase-shift detection technique would not truly utilize the Doppler-effect in its detection of phase shift. Since there is no flow involved within static compartments, then there is no Doppler-effect possible. Within vessels, however, the desire is to cancel out any effect of flow and motion artifact. Therefore, while phase-shift may still be a measurable quantity, the Doppler-effect would not be applicable in either the vascular setting or the static compartment setting.

The single transducer transit time and phase-shift detection methods may be more suitable for measurements of static compartments or hollow organs within which flow is not a significant factor. They may be less suitable for vascular pressure measurements where they cannot easily cancel out noise caused by flow.

IR, RF, and/or Laser technology may also be used in transducer design for the single or two-transducer methods. The arrangement of the sender and receiver transducers would be as in FIGS. 1 and 2. Sensor function could be enhanced with the use of Laser technology with Fiber Bragg Gratings (FBG's) tuned to a specific US frequency. FBG Laser may be especially useful in sensor design due to its capability of sensing high frequencies and its resistance to RF interference.

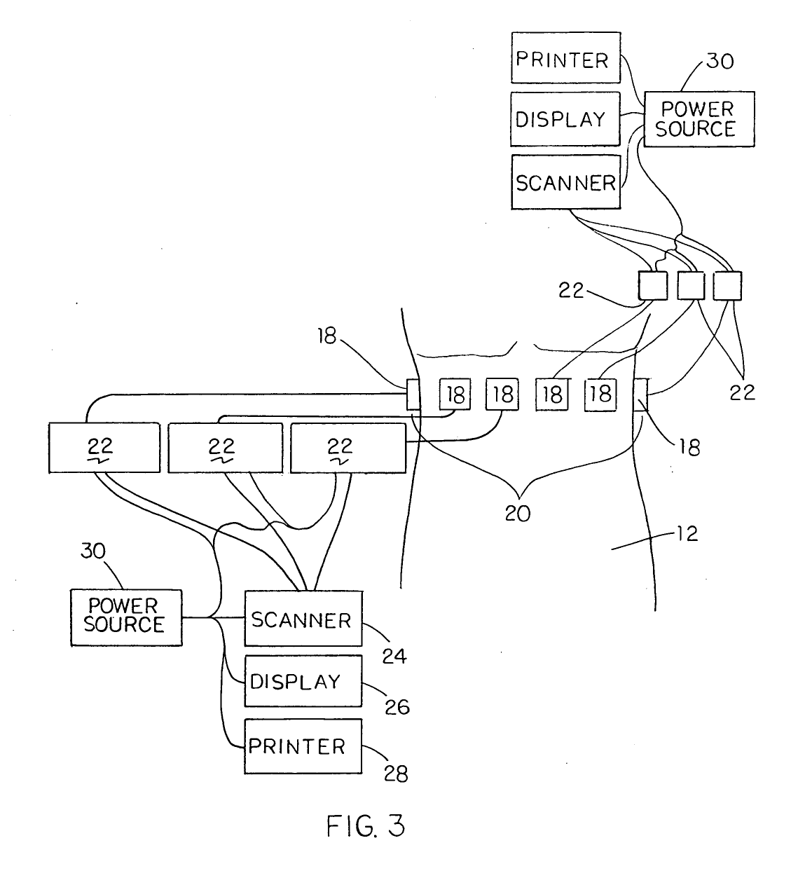

A third type of arrangement for the transducers utilizes three or more transducers, one sender and two receivers arranged in the order, receiver-sender-receiver, as they lay longitudinally over the vessel. Again, these transducers could be of piezoelectric design or could use any of the other advanced technology above described. The two receiving transducers would clock the US wave front as it passes upstream and downstream from the centrally located sending transducer. The velocity values would be summed in order to cancel out the effect of blood flow and to separate it from the effect produced by pressure fluctuations. Like the two-transducer technique, this technique—given very specific placement of the transducers and chosen frequencies—would also take advantage of the fact that the attenuation coefficients of biologic tissues differ from that of biologic fluids. However, accuracy would likely not be as precise as with the two-transducer method since the wave paths upstream and downstream do not cross the same section of vessel, and thus cancellation of turbulence-induced signal variations may not be as effective. See FIG. 3.

Each of the sensors 14, 16 and 18 and each of the monitoring devices 10 of the invention illustrated in FIGS. 1-3 are connected to a computer that is programmed with recognition and analysis software. Depending upon the function of the sensor 14, 16 or 18, i.e., whether the sensor is a sender, a receiver, or both, the computer software will differ as to each recognition and analysis computer 22 to receive the signal from its individual sensor 14, 16, 18 and convert the same into a measurement of arterial and venous blood densities and blood flow velocities, blood pressure, pulse rate, vascular resistance, cardiac output, pressure pulse wave velocity, and the like. The display will include both instantaneous measurements and a plot of each measurement versus time.

Scanners 24 are provided to scan each of the computers 22 sequentially from about 10 to about 100 times per second, depending upon the particular clinical application. Each of the scanners would be operatively connected to a display 26 that would display the data from each of the sensors 14, 16, 18 of each of the measurements, in the form of both instantaneous measurements and the historical trends of each measurement. The display would be combined with a selection switch by which each measurement and trend could be selectively displayed.

Attached to each display would be a printer 28 which would print out current vital signs and a continuous record of highest, lowest, and trends of each measurement, as well as trends for each patient and each location of a sensor 14, 16, 18.

Each of the sensors 14, 16, 18, each of the recognition and analysis computers 22, each of the scanners 24, each of the displays 26, and each of the printers 28 are connected to a power source 30.

In the single sensor device 10 illustrated in FIG. 2, the sensor 16 is connected to a single recognition and analysis computer 22 which is connected directly to a display 26 and to a printer 28.

Precise measurement of biologic fluid density is the critical step in ensuring accuracy by way of continuous calibration for the device using any of the above methods. Also, such continuous and precise monitoring of blood density would be extremely useful in the diagnosis and treatment of trauma patients and any other malady involving rapid or profound blood loss or physiologic fluid shifts. See device 100.

Morbid obesity would likely make this device unusable as it would increase signal attenuation and would therefore make readings very difficult. There would be a marked decrease in the signal-to-noise ratio in patients with thick layers of adipose tissue. When working within normal physiologic blood pressure ranges, period calibration of the device using a conventional sphygmomanometer would solve the problem of accuracy in most situations where body habitus interferes with the normal function of the device. However, in the non-obese patient, even in cases where calibration is not possible, such as a profound hypotension or cardiac arrest, accurate readings may be attainable with the device.

Another challenge would be the design of a stable transducer-to-skin interface acoustic conductive medium. The interface must remain fixed in position for a number of hours. It must be comfortable to the patient, and provide reliable ultrasound conduction.

In summary, the vascular application of the device would be capable of accurately and continuously measuring arterial and venous blood pressures, pulse rate, blood density, and blood flow velocity, and it would be capable of calculating peripheral vascular resistance. When the venous system and interstitial space are monitored, the state of hydration can be assessed. When applied to the chest, then pulmonary, central venous, pleural space and cardiac monitoring may also be possible. The device may have many other uses, including the measurement of compartment, ocular, intra-abdominal, intracranial, and specific organ pressures. In addition, the device could be mated to other more conventional equipment, e.g., measuring oximetry and temperature.

### Device 100

Another version of the new and improved noninvasive blood density measurement device 100 is a simpler form of device 10 of the invention for the purpose of supplying in vivo blood density information for medical monitoring and research. The function of device 100 is the same as that of device 10, except that ultra-precision is not required. As with device 10, the goal with device 100 is the noninvasive in vivo measurement of blood density. However, it will not have the necessary precision to detect the minute density fluctuations which represent pulse pressure. Therefore the device requires somewhat less sophistication.

The primary goal of monitoring blood density is to detect fluid shifts within the body. Also, because hematocrit is the main contributor to the density of whole blood, then both device 10 and device 100 are continuous noninvasive hematocrit monitors. They could, therefore, both be used to monitor multiple parameters in critically ill or injured patients or be used to spot check patients for blood disorders.

Within the practice of nephrology, the monitoring of blood density during dialysis is well known to be important as it is used to predict and preempt sudden onset of hypotension. However, the relevance of blood density values and trends as they relate to the status of critically ill or injured and potentially unstable “critical” patients is not well known. Under the current state of the art in blood density measurement, comprehensive research on the clinical relevance of blood density is not possible. Device 100 is needed so that such clinical relevance, or lack thereof, can be discovered.

Currently the state of the art in blood density measurement is practiced using only extracorporal methods. One method uses frequent blood sampling and subsequent laboratory analysis. Another less precise method uses a continuous optical device during hemodialysis which clamps onto the dialysis tubing and measures the concentration of the extracorporal blood. Continuous blood density monitoring is currently unavailable for patients who are not undergoing hemodialysis. Frequent blood sampling, although precise, is labor intensive, expensive, and impractical. There is a need for a tool such as device 100 which measures blood density conveniently, continuously, noninvasively, and in vivo. In addition, device 100 may be capable of providing continuous data relating to arterial and venous blood flow velocities, extravascular fluid stores, and analogs of vascular resistance and cardiac output.

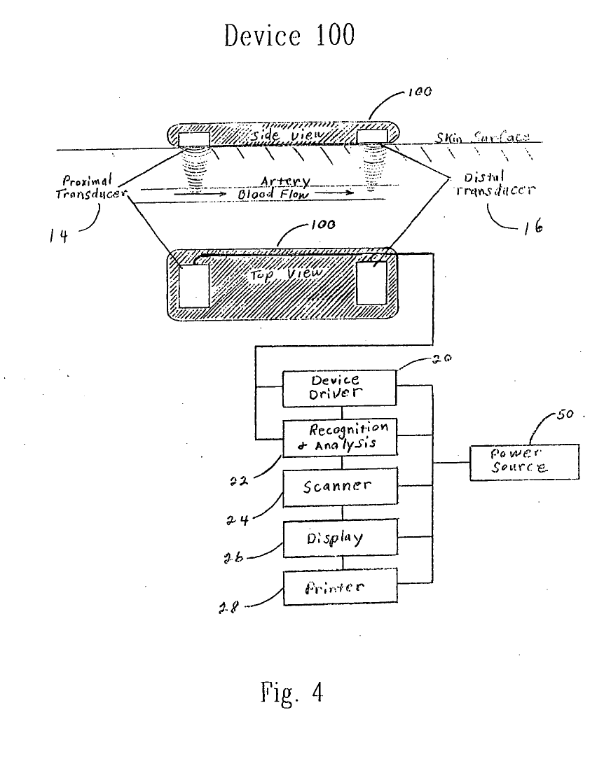

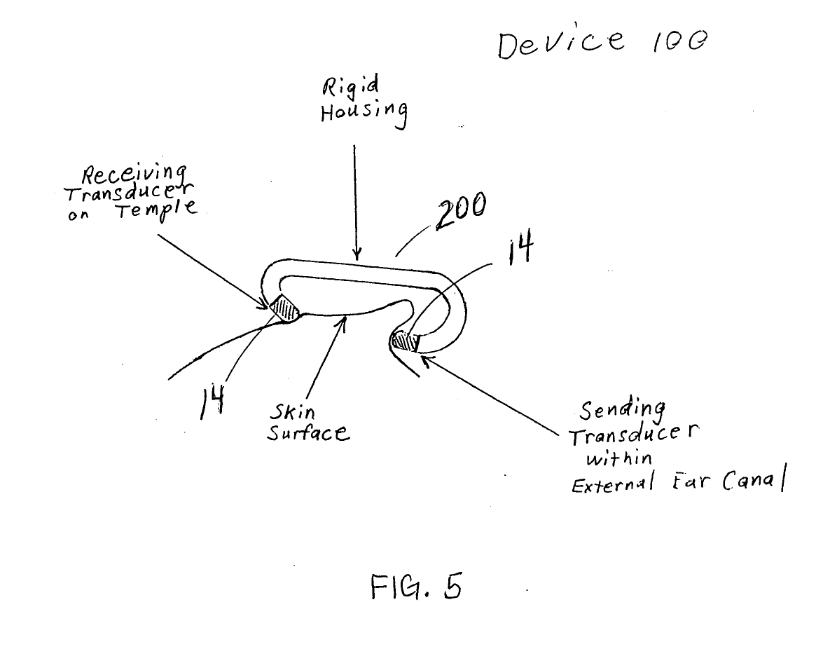

This description of device 100 is also an addendum to the description of device 10. Most of the technical aspects of device 10 are identical to that of device 100, and therefore, the text of this description applies fully to that of device 10. FIGS. 4 and 5 illustrate one possible form of device 100 and device 10 of the invention. Although the basic function of the two devices is almost identical, the goals of precision and clinical application differ and would dictate certain technical variations.

### Physiological Basis for Measurement of Blood Density in Critical Patients

During severe physiological stresses there are significant alterations in blood density as the blood becomes more concentrated or more dilute. These alterations occur as a result of transcompartmental fluid shifts that, in turn, are caused by physiologic compensations in for form of osmotic or hydrostatic effects. Significant fluid shifts—and thus dynamic changes in blood density—occur during shock from any cause including hemorrhage, sepsis, spinal cord injury, toxins, and cardiogenic causes. Less significant, but still noteworthy, are fluid shifts and blood density changes that occur during more ordinary clinical situations such as orthostasis (1), dehydration and rehydration, various pharmacological therapies, and weightlessness. Therefore, the measurement of blood density and its trends may thus become an important tool for ruling out certain causes of syncope and dizziness.

When the condition of low intravascular volume or pressure occurs, physiologic compensations are triggered in an attempt to maintain blood volume and thereby blood flow to the vital organs. Under these conditions the vascular system osmotically draws fluid into the blood from the extravascular space. This results in dilution of the blood and a drop in blood density (hemodilution). For example, hemorrhage results in rapid fluid movement from the extravascular to the intravascular space and thereby causes hemodilution. (2) The vascular system is, in effect, “borrowing” fluid from the tissues in order to preserve blood volume and flow.

Similarly, an infusion of IV fluids will initially cause hemodilution and a drop in blood density. However, the dilution from IV fluids will not persist if the blood volume and osmotic and hydrostatic pressures remain adequate, because the fluid will eventually migrate from the blood to the extravascular space (if it is not first lost through renal excretion or other insensible losses). The vascular system thus gives back fluid that it may have once “borrowed” from the tissues, and the blood density or concentration will drift back toward a more normal range.

Certain catastrophic vascular effects are triggered by prolonged or severe shock, sepsis, burns, crush injuries, and toxins. The result of these effects is capillary damage and leak. This leaking causes fluid to shift from the blood to the extravascular space, and thus results in a blood volume decrease accompanied by a blood density increase (hemoconcentration). Since diuretics and blood transfusions effectively cause hemoconcentration, and IV fluids cause hemodilution, then device 100 could also become a useful tool in the monitoring and management these types of therapeutic interventions.

The clinical course for severely ill or injured patients is typically very dynamic. As the disease process takes its course, the physician then responds with aggressive treatment using surgical techniques, vasoactive drugs, IV fluids, and blood products. Multiple events take place in rapid sequence, and each has its own effect upon blood density. In such dynamic situations, the interpretation of blood density values and trends would be complex. Clinical studies must be done in order to define the parameters for use of device 100.

Since blood density is already known to be an important indictor to observe during hemodialysis, it is reasonable to assume that there is also a relationship between the clinical course experienced by the critical patient and the magnitude and rapidity of the changes in blood density. It is likewise reasonable to assume that there are physiological limits to blood density and that high and low extremes are not compatible with life. Therefore, blood density monitoring may be as important in the management of any critical patient as it is in the management of a hemodialysis patient.

The density of blood is determined by multiple factors, the most influential of which are the hematocrit and serum protein level. Other determining factors are pressure, temperatures, and dissolved sugars, salts, and gases. Since device 100 is not designed for ultra-precision as is device 10, the blood pressure contribution to density values will be negligible. For purposes of simplicity during earthbound research, pressure would be assumed to be fixed at one atmosphere. Also, since it has a significant effect upon density, a calibration or correction for temperature must be accomplished.

The device would be useful in any medical setting where care is provided for critical patients. This would include such settings as emergency departments, intensive care units, surgical or post-operative areas, burn units, hemodialysis units, military mash units, and battlefield settings. It would also be useful in aerospace research settings. There are significant transcompartmental fluid shifts that occur in the zero gravity environment. (3, 4)

In order to ultimately find its niche among the tools of clinical medicine, the device must initially serve the purpose of research tool. To date it appears that there has been very little research done and in the area of blood density and its correlation to the clinical status of critical patients. However, the few studies that have surfaced do seem to indicate that such monitoring would probably be useful, provided that convenient method of measurement is available. There is currently no existing device for monitoring blood density in vivo and noninvasively on a continuous basis.

### Device Function—Another Version

The function of device 100 depends upon in vivo sound speed measurement within blood. Its basic function is identical to that of device 10, but with less required precision. Acoustic transit time (time-of-flight) is measured using the “pitch-catch” method between proximal and distal transducers in both upstream and downstream directions simultaneously. Another option would to replace one transducer with a reflector. The remaining transducer would act as both sender and receiver, and would clock total time-of-flight. Either device would be applied to the body wherever the best arterial and venous signal can be obtained. These time-of-flight measurements would be then be used to calculate acoustic velocities upstream and downstream. Readings would be taken from both arterial and venous blood and then the data from each or averaged data from both would be fed into a microprocessor where it would be converted to blood density and flow values.

When using the time-of-flight method to obtain blood density readings, the effect of blood flow velocity upon time-of-flight must be negated. Although the condition of “static” blood is not possible under most in vivo circumstances, a correction can be made for the effect of flow upon the measurements. BY using the upstream and downstream velocity values, a calculated “static equivalent” acoustic velocity can be obtained. The effect of flow is thus cancelled out mathematically by dividing the sum of the upstream and downstream acoustic velocities by two. The result is the acoustic velocity equivalent as if it were measured within static blood.

In practical terms it may be very difficult to separate the arterial and venous signals since, in the circulatory system; the arteries and veins are usually paired and located in close proximity to one another. The signal would thus be essentially already averaged along with the portion of the signal attributable to capillary blood.

Based upon the UNESCO equation for sound speed in water, this resulting “static equivalent” acoustic velocity value has a direct mathematical correlation with blood density as discussed in the description of device 10. The UNESCO equation—which was developed for the study of sound speed in seawater—can be used to substantiate this method, since blood, like seawater, is simply water with certain substances in solution or suspension.

Blood flow velocity would be calculated by subtracting the upstream from the downstream acoustic velocity and dividing by 2. Both venous and arterial flow velocities can be calculated in this fashion. This time-of-flight method of blood flow velocity determination differs from that of the Doppler method, since its operation depends upon sound-speed measurements and not phase-shift data.

Since the device senses blood flow in both directions, it would be able to differentiate venous from arterial flow. In terms of practical function, the fact that it is sensing blood flow assures that the device is indeed reading the density within intravascular fluid and not within extravascular fluid.

Interestingly, device 100 may also be capable of providing a measurement of acoustic velocity within the extravascular space. This would be useful in monitoring the body's stores of extravascular fluid, i.e., hydration status. Sound speed should change proportionately with the fluid “saturation” of the extravascular tissue.

Another method of assessing the dynamic status of the extravascular fluid would be to monitor the difference between arterial and venous blood density values. This may present a challenge if the arterial venous blood density measurements can not be distinguished one from the other, but the data obtained from this method would contain relevant information relating to the movement of fluid into and out of the extravascular space. For example, when the arterial blood density is found to be higher than the venous blood density, the conclusion might be made that physiologic compensation is underway and that fluid is moving from the extravascular space into the blood, such as might be seen during blood loss. This differential arterial-venous blood density value would probably be detectable prior to any significant alteration in whole blood density and would serve as a very sensitive and contemporaneous real time gauge of intravascular-extravascular fluid movement.

By applying existing state of the art methods of measure, this device could also sense pulse-wave velocity by clocking the pulse-wave as it passes by the two transducers. Pulse-wave velocity is the speed of the arterial pressure wave as it propagates from the heart to the peripheral tissues. It can be used to estimate cardiac output when adjusted for patient age. An analog of cardiac output could most likely be calculated from mean arterial blood flow and pulse-wave velocity. An analog of vascular resistance could also theoretically be calculated if mean arterial and venous pressures and blood flow are known. The actual analog values of cardiac output and vascular resistance obtained in this manner would be useful only for trending. (5)

Another already existing method of measuring pulse-wave velocity involves mating the device with an electrocardiogram (EKG) electrode and measuring the time from the QRS impulse to the arrival of the pulse-wave at the device. Also, contour analysis of the pulse pressure wave is a method currently being used to estimate cardiac output. These methods seem to be gaining some validity within the research literature as being reliable for trend monitoring of cardiac output. (6)

Like device 10, device 100 would digitally display the instantaneous arterial and venous blood density and blood flow velocities, the analogs of cardiac output and vascular resistance, and their trend lines as well as rate of change.

The acoustic frequency range to be used would vary with the desired separation distance between transducers. Both are yet to be determined. As with device 10, the electronics required would include a device driver 20 that controls each transducer by triggering impulses at a rate between 10 Hz and 100 Hz. Each transducer would act as both sender and receiver and would be connected to a signal processing computers 22 that would note time-of-flight for each impulse and then calculate the dimensions and location of the vessels and arterial and venous blood density and flow velocities. The receivers would also send pulse-wave velocity data to the computers 22 for signal processing and calculation of vascular resistance and cardiac output analogs. Scanners 24 would collect data from the computers and transmit it to the display 26 and printer 28. A power source 50 would be connected to all components.

The housing for the transducers (FIG. 4), which would most likely be constructed from medical grade epoxy or silastic, would fix the distance between the two transducers and set the proper angle to the skin in order to create the most optimal signal for processing. The requirements for housing style might differ depending upon the anatomic location to be monitored. For use on the arm, for example, the housing must be as flat as is practical and secured in some fashion. Testing must yet be performed in order to find the most optimal transducer separation, angle, and frequencies, as well as the best type of acoustic conductive medium, arm band style, etc.

There would possibly be a need to tune (power, transducer wavelength, transducer triggering frequency, and beam spread or scatter) the ultrasound beam in such a way as to effectively maximize the intravascular acoustic travel distance (the distance that the sound wave actually travels within the blood vessel on its path to the receiving transducer). Such tuning might take the form of a transducer array possibly requiring the use of more than one frequency.

As with device 10, and for the sake of precision, the operational goal of device 100 is for the sending transducer to create a shock wave “click,” and to clock its transit time within the blood to the receiving transducer. Since sound waves travel in all directions from their point of origin, it would be difficult to know the exact length of the intravascular sound wave path. However, fixing the transducer separation by using rigid housing and using a technique such as Time-Reversal Mirrors should define the wave path well enough to accomplish the desired level of precision. Maximizing and closely defining the length of the sound wave path would be a crucial step for the accurate determination of intravascular sound speed. Detection and timing of the first arrival wave would indicate the transit time for the most direct path between transducers. Since sound speed is higher in blood than in the surrounding tissues, then this first arrival wave would be considered to have passed through blood.

Therefore, both sending and receiving transducers may need to be tuned (focused or defocused) to maximize the path within the blood. Tuning may need to be individualized for each patient and for each anatomic location. The induced intravascular “click” would emanate in all directions from its point of origin within the vessel. Much of the resulting wave energy would then travel through the blood (arteries, veins, and capillaries) and be detected by the receiving transducer. The effect of this tuned shock-wave technique would be to maximize the intravascular travel distance of the acoustic wave. It would also improve signal-to-noise ratio for the time-of-flight impulse.

The remainder of the wave energy would reflect and/or scatter and would ultimately also be detected by the receivers. The data collected from the reflections would reveal the status of the extravascular fluid balance, and would also be used to determine the size and location and the selected vessel. This data would in turn be fed into the signal processors and could be used to guide the tuning of the ultrasound beam. By applying signal processing techniques, the true intravascular signal would be separated from the signals resulting from reflections and scatter.

For device 10 this technique of tuned input and detection would likewise be applied for the purpose of signal enhancement and precision, even though input frequencies and transducer types might differ.

The type of transducer to be used in device 100 is also yet to be determined. Common piezoceramic transducers may work well, but in order to assure the proper brevity of the input impulse “click”, other types of transducers may be required, including but not limited to, polymer, piezo, laser, infrared, radio frequency, Fiber-Bragg laser receivers, and hybrid transducers.

### Device Function—Still Another Version of Device 100

Although sound velocity measurements would ideally be made longitudinally through a vessel, in practical terms this might be very difficult to do non-invasively. Sound waves prefer to travel along straight paths. Therefore the most practical body location chosen may involve the acoustic wave crossing a vessel perpendicularly. If high enough frequencies are used (1 to 20 MHz), then the desired precision might still be accomplished. The vessels adjacent to the external ear may be amendable to such a monitoring method. Other locations such as the perioral and post-auricular arteries might also be used.

A specific example is the superficial temporal vessels, located just anterior to the tragus of the external ear. In this case the vessel may be monitored by placing the sending transducer against the anterior wall of the external ear canal just behind the tragus. The receiving transducer would then be placed against the skin anterior and superior to the tragus, positioning the vessel between the two transducers. The sender would emit its impulse directly towards the receiver across the vessel. Continuous time-of-flight measurements would be continuously corrected for temperature changes and converted into blood density values. If the separation between transducers can be maximized, and if frequencies used are high enough (500 KHz to 100 MHz), then desired precision might be accomplished. See FIG. 5. Temperature correction may be accomplished by incorporating a temperature probe into the device just adjacent to the transducer or with the use of a dual-mode oscillator crystal, which has the characteristic of self-temperature-sensing.

In order to maintain accuracy of sound velocity readings and thus blood density measurements, the device would incorporate a temperature probe and also electronics for continuously monitoring the mechanical separation between transducers. Since movements such as chewing and talking may change the separation of the transducers, the device could instantaneously adjust for the change and recalculate sound speed based upon the new distance. In all practicality, the separation between the transducers or transducer and reflector should be fixed by using a rigid housing or include a monitoring mechanism for measuring changes in the separation distance between the transducers or reflector. This would apply to both device 10 and device 100. Also, with either device, it may be relatively easy to incorporate an oximeter. The application of a small array at the receiver may provide vessel diameter information and thus an analog of pulse pressure.

In summary, device 100 may provide the following continuous data from a stable platform overlying a vessel near the ear:
1. Blood density
2. Temperature
3. Oximetry
4. Pulse rate
5. Pulse pressure analog

### REFERENCES

(1) Hingohofer-Szalkay, J E Greenleaf. Continuous monitoring of blood volume changes in humans. J Appl Physiol. 1987; 63: 1003-7
(2) Hinghofer-Szalkay H. Continuous blood densityometry: Fluid shifts after graded hemorrhage in animals. Am J Physiol 1986; 250 (Heart Circ. Physiol. 19): H342-50
(3) Hinghofer-Szalkay H., Koenig E, Schmied J, Heimel H. A new principle for dynamic fluid shift investigations in astronauts. Proc 4^{th} Eur Symp Life Sci in Space: esa sp-307, 129-132, 1990.
(4) Watenpaugh, D E, and Hargens A R. The cardiovascular system in microgravity. In: Handbook of Physiology. Environmental Physiology Bethesda, Md.: Am Physiol. Soc, 1996, sect. 4, vol. I, chapt. 29, p. 631-674
(5) Quick, C M, Berger D S, and Noordergraaf A. Apparent arterial compliance. AM J Physiol Heart Circ Physiol 274: H1393-H1401, 1998
(6) S. M. Tibby and I. A. Murdoch. Monitoring cardiac function in intensive care. Archives of Disease in Childhood 2003; 88:46-52

While specific embodiments of the invention have been shown and described herein for purposes of illustration, the protection offered by any patent which may issue upon this application is not strictly limited to the disclosed embodiment; but rather extends to all structures, steps and arrangements which fall fairly within the scope of the claims which are appended hereto:

What is claimed is:

1. A blood monitoring device comprising a body having a proximal and distal transducers spaced apart and connected to both device drivers and recognition and analysis devices, a power source connected to both said device drivers and said recognition and analysis devices, said device driver also connected to said recognition and analysis devices.

2. The device of claim 1 wherein the transducers are tuned to a point within a human being's blood.

3. The device of claim 2 wherein said transducers are tuned to ambient atmospheric pressure and temperature.

4. The device of claim 1 wherein both proximal and distal transducers include a plurality of transducers.

5. The device of claim 1 wherein said plurality of transducers are tuned to different frequencies.

6. The device of claim 1 wherein said proximal and distal transducers are both senders and receivers.

7. The device of claim 1 wherein said transducers may be chosen from the group of transducers consisting of piezoceramic transducers, import impulse click transducers, polymer piezo transducers, laser transducers, infrared transducers, radio frequency transducers, Fiber-Bragg laser receivers, hybrid transducers and combinations thereof.

8. The device of claim 1 wherein said device drivers is chosen from the group of device drivers consisting of impulse generators, wave generators, and electronic switches.

9. The device of claim 1 wherein said recognition and analysis devices is chosen from the group of recognition and analysis devices consisting of scanners, displays, computers, printers, recorders, senders, receivers, and combinations thereof.

10. The device of claim 1 wherein proximal and distal transducers may be adjusted as to operational frequency.

11. The device of claim 1 wherein said proximal and distal transducers have frequencies ranging from about 500 KHz to about 100 MHz

12. The device of claim 1 wherein said recognition and analysis devices measures time of flight, arterial density, venous density, hematocrit, flow velocity, venous flow velocity, arterial flow velocity, analogs of vascular resistance, analogs of cardiac output, signal to noise ratio, and combinations thereof.

13. The device of claim 1 wherein said device may be adjusted for ambient temperature and varying distances between said distal and proximal transducers.

14. The device of claim 1 wherein said proximal and distal transducers are on opposite sides of a blood vessel.

15. The device of claim 14 wherein said blood vessel is a vein.

16. The device of claim 14 wherein said blood vessel is an artery.

17. The device of claim 14 wherein the path over which the sound waves travel between the proximal and distal transducers are generally perpendicular to said blood vessel.

18. The device of claim 1 wherein the frequency of the transducers is between 5 and 10 mhz.

19. The device of claim 14 wherein said proximal and distal transducers are on opposite sides of the periorral arteries.

20. The device of claim 1 wherein said proximal and distal transducers are on opposite sides of the post auricular arteries.

21. The device of claim 1 further comprising an oximeter.

22. The device of claim 1 wherein said recognition and analysis devices emit a continuous record of data relating to the measurements of the group of measurements consisting of blood density, hematocrit, temperature, arterial and venous blood pressure, pulse rate, blood flow velocity, and pulse pressure analog, or combinations thereof.
